(19) United States
(12) Patent Application Publication
(10) Pub. No.: US 2006/0252738 A1
(43) Pub. Date: Nov. 9, 2006

(54) Method for preparing and using water-based steroid pheromone compositions

(75) Inventors: Norman Avelino, Richmond (CA); Jean Pierre Lafontaine, Ladner (CA)

Correspondence Address: OYEN, WIGGS, GREEN & MUTALA LLP;480 - THE STATION, 601 WEST CORDOVA STREET, VANCOUVER, BC, V6B 1G1, CA

(73) Assignee: Phero Tech Inc., Delta (CA)

(21) Appl. No.: 11/123,032

(22) Filed: May 6, 2005

(51) Int. Cl. A61K 31/57; A61K 31/185

(52) U.S. Cl. 514/177; 514/553

(57) ABSTRACT

This invention relates to methods of formulating steroid pheromones as novel stable emulsions. These emulsions can be used for administration of the steroid pheromones to living organisms such as humans or pigs in place of current organic solvent or pressurized aerosol formulations that present hazards in both shipping and application. Uses relating to pigs are in stimulation of sexual maturation, diagnosis of the onset and timing of oestrus in female pigs, and inducing boars to accept dummy sows. The water-based emulsions can include 5α-androst-16-en-3-one and 3α-hydroxy-5α-androst-16-ene for administration to pigs. The use of water-based emulsions eliminates the need for formulation of the steroid pheromones in organic solvents, delivery of these pheromones as pressurized aerosols, and following the precautions required to reduce hazards during shipping and handling. The water-based emulsions allow for treatment of living organisms where a liquid organic solvent formulation or aerosol of the pheromones might be harmful to the organism or a substrate.

### FIELD OF THE INVENTION

This invention relates to methods of formulating steroid pheromones as novel stable emulsions. These emulsions can be used for administration of the steroid pheromones to living organisms such as humans or pigs in place of current organic solvent or pressurized aerosol formulations that present hazards in both shipping and application. Uses relating to pigs are in stimulation of sexual maturation, diagnosis of the onset and timing of oestrus in female pigs, and inducing boars to accept dummy sows.

### BACKGROUND OF THE INVENTION

Steroid pheromones range from being insoluble to sparingly soluble in water (Hoover, 1975). Various organic solvents must be used to extract steroid phero-mones from mammalian subjects. These include methanol (Brooksbank et al. 1974; Nixon et al. 1988), ethanol (Cutler et al. 1987, 1989, 1990, 1992; Preti et al. 1987), diethyl ether (Patterson 1968a,b; Gower et al. 1970), acetone (Patterson 1968b; Brooksbank et al. 1974), and ethyl acetate (Nixon et al. 1988; Kwan et al. 1992).

Solvents used to present steroid pheromones such as androstene steroids to mammalian subjects include ethanol (Kirkwood et al. 1983, Filsinger et al. 1984; Benton and Wastell 1986; Cutler et al. 1992), diethyl ether (Kirkwood et al. 1983), chloroform (Cowley and Brooksbank 1991), isopropyl alcohol (Shinohara et al. 2000, 2001), and propylene glycol (Jacob and McClintock 2000). Accordingly, Melrose et al. (1972) teach that androstenone “is introduced into the environment of the female pig with an inert carrier comprising a perhalogenated lower alkane aerosol propellant”, or “a volatile organic solvent”.

Two C₁₉-16 unsaturated androgen steroids, 5α-androst-16-en-3-one (androstenone) and 3α-hydroxy-5a-androst-16-ene (androstenol), are found in the testes, saliva and fat of male pigs (boars) (Patterson 1968a,b). These and other C₁₉-16 unsaturated steroids also occur in the blood and urine of boars (Gower et al. 1970). Similarly several 16-androstenes are found in the axillary secretions, urine, semen and blood of humans (Brooksbank et al. 1974; Claus and Alsing 1976; Nixon et al. 1988; Kwan et al. 1992). In addition, Δ4,16-androstadien-3-one (androstadienone) was isolated from human skin (Preti and Wysoki 1999). Two androgen steroid sulfates, 17-oxo-5α-androstan-3α-yl sulfate (androsterone sulfate) and 17-oxo-5α-androsten-3β-yl sulfate (dehydroepiandrosterone sulfate) are present in high concentrations in human axillary secretions, suggesting that such sulfates may serve as precursors of 16-androstenes (Labows et al. 1979; Preti et al. 1987).

In pigs, androstenone and androstenol, administered as pressurized aerosol sprays in organic solvents, have been shown to function in place of boar saliva in inducing an immobile mating stance in sows that have reached oestrus (Melrose et al. 1971). A composition comprised of either or both substances provides a diagnostic tool for determining the time of maturation of young female pigs (gilts) and the time of oestrus in sows (Melrose et al. 1972). Reed et al. (1974) found that as diagnostic tools, andrsost-4,16-dien-3-one, 5β-androst-16-en-3-one, and blends at different concentrations of androstenone and androstenol, were comparable in bioactivity to androstenone, but that 5α-androstan-3-one or 3β-hydroxy-5α-androst-16-ene were less effective than androstenone. There is a suggestion that exposure to 16-androstenes may accelerate the onset of puberty in gilts (Kirkwood et al. 1983; Glei et al. 1984). Estienne and Harper (2001) advise spraying a product that contains a 16-androstene onto a dummy sow to train a naive boar to mount a dummy sow for semen collection.

Jennings-White et al. (2000a,b) and Berliner and Monti-Bloch (2001) describe general methods of formulating steroid pheromones for administration to humans using a carrier (“water, saline, aqueous dextrose, glycerol, ethanol and the like”), and adjuvants (“wetting or emulsifying agents” and “surfactants”) to “form a solution or suspension.” They do not, however, describe any formations or any uses for such solutions or suspensions, nor do they acknowledge that such a composition could be applied without using an organic solvent or a propellant. Rather, they indicate that “topical application of a volatile liquid composition to the skin” involves an organic solvent formulation that “will usually contain an alcohol such as ethanol or isopropanol”. Moreover, for aerosol administration, they stress the need for a propellant formulation maintained at an “elevated pressure until released by action of a valve.”

All commercial androstene steroid products used on pigs since 1972 have followed the teachings of Melrose et al. (1972), despite the fact that products comprising organic solvent solutions of steroid pheromones in pressurized aerosol cans present an explosive hazard, and are subject to rigorous restrictions with regard to packaging and shipping. Because of their flammability they also require the adoption of stringent safety precautions in storage and use (Antec International 1996). Exposure of mammalian skin to flammable organic solvents may be harmful, and certain substrates, for example some plastics, may be damaged.

### SUMMARY OF THE INVENTION

The invention is directed to a method of formulating a water-based steroid pheromone containing emulsion comprising: (a) mixing a steroid pheromone with a suitable hydrotrope to form a first mixture; (b) mixing a suitable hydrotrope with a suitable surfactant to form a second mixture; and (c) mixing the first mixture with the second mixture while adding a suitable amount of water to form the water-based emulsion.

The emulsion can be a micro-emulsion. The steroid pheromone can be an androstene pheromone selected from the group consisting of one or more of: (a) a C₁₉-16 unsaturated androgen steroid, including 5α-androst-16-en-3-one, 3α-hydroxy-5α-androst-16-ene, and 5β-androst-16-en-3-one; (b) a C₁₉-4,16 unsaturated androgen steroid, including Δ4,16-androstadien-3-one.

The hydrotropes can be a combination of mono- and di-alcohols. The hydrotropes can be selected from the group consisting of methanol, ethanol, propanol, butanol, pentanol, hexanol, propanediol, butanediol, pentanediol and hexanediol, and isomers thereof, and any combination of two or more of the hydrotropes.

The surfactant can be selected from the group consisting of alkylated aryl sulfonic acids, including dodecyl benzene sulfonic acid; amine-neutralized alkylated arylsulfonates, including dodecylbenzene sulfonic acid neutralized with monoisopropyl amine; ethoxylated alkylphenols, including nonylphenols ethoxylated with 9 moles of ethylene oxide; and unsubstituted fatty esters of a polyoxyalkated sorbitan, including sorbitan mono-oleate ethoxylated with 20 moles of ethylene oxide.

The preferred proportions by weight (% w/w) can be: mono-alcohol 0.05-0.2, di-alcohol 1-10, steroid 0.0001-0.1, surfactant 0.1-0.5, and water quantity sufficient to bring the composition to 100% w/w.

The invention is also directed to a composition comprising a water-based emulsion formed from a steroid pheromone, one or more hydrotropes, a surfactant and water.

The invention is also directed to a method of applying a steroid pheromone emulsion composition to a substrate comprising dispensing the emulsion composition according to the invention as an effective amount of an aerosol, a liquid, droplets, a paste or a treated inert substrate.

The composition can be an aerosol dispensed from an aerosol dispensing device. The device can be a hand-held atomizer.

The emulsion composition in one specific embodiment can be comprised of one or more steroid pheromones selected from the group consisting of C₁₉-16 unsaturated androgen steroids, 5α-androst-16-en-3-one, 3α-hydroxy-5α-androst-16-ene, 5β-androst-16-en-3-one; C₁₉-4,16 unsaturated androgen steroids, including Δ4, 16-androstadien-3-one; hydrotropes selected from the group consisting of methanol, ethanol, propanol, butanol, pentanol, hexanol, propanediol, butanediol, pentanediol, hexanediol, and any isomers thereof, any combination of two or more of said hydrotropes, surfactants selected from alkylated aryl sulfonic acids, including dodecyl benzene sulfonic acid; amine-neutralized alkylated arylsulfonates, including dodecylbenzene sulfonic acid neutralized with monoisopropyl amine; ethoxylated alkylphenols, including nonylphenols ethoxylated with 9 moles of ethylene oxide; unsubstituted fatty esters of a polyoxyalkated sorbitan, including sorbitan mono-oleate ethoxylated with 20 moles of ethylene oxide. The composition can be a micro-emulsion.

The aerosol, liquid, droplets, paste, or treated inert substrate can be introduced in an effective amount into the environment of a mammal. The mammal can be a pig.

The aerosol treatment can be used in effective amount as an aid in diagnosing the onset of oestrus in gilts. The aerosol treatment can be used in effective amount as an aid in diagnosing the timing of oestrus in sows. The aerosol, liquid, droplets, paste, or treated inert substrate treatment can be used in an effective amount as an aid in accelerating the onset of puberty in gilts. The inert substrate can be a dummy sow used during collection of semen from a boar.

### DETAILED DESCRIPTION OF THE INVENTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification is to be regarded in an illustrative, rather than a restrictive, sense.

We have discovered that steroid pheromones, including androstene pheromones, can be formulated in water rather than organic solvents, and can be delivered without using a propellant, to the treatment mammals, including pigs. The products are safe to use, require fewer restrictions in packaging and shipping, and are potentially less damaging to exposed organisms or substrates than formulations in organic solvents.

Past research and practice has taught that androstene steroid pheromones, including 5α-androst-16-en-3-one (androstenone) and 3α-hydroxy-5α-androst-16-ene (androstenol), must be dissolved in organic solvents and formulated using a pressurized propellant for delivery as an aerosol to pigs. Employing such a formulation delivered as an aerosol has enabled the use for many years of these pheromones to diagnose the onset of sexual maturity in gilts and the timing of oestrus in sows. Other uses include treatment of a dummy sow to train a naive boar to accept and mount the sow for semen collection, and induction of accelerated reproductive maturation in gilts.

Compositions involving organic solvent solutions of steroid pheromones in pressurized aerosol cans can present an explosive hazard, and hence are subject to rigorous restrictions with regard to packaging and shipping, and the implementation of stringent safety precautions in storage and use.

Specifically, we have discovered that steroid pheromones, including androstene pheromones, can be successfully formulated as water-based emulsions, specifically micro-emulsions, that are stable at room temperature, and unexpectedly remain stable through successive cycles of freezing and thawing. These formulations require neither organic solvents nor pressurized propellants for delivery, and thus present no hazards that would make them subject to the same restrictions and precautions as formulations involving organic solvents and pressurized aerosol cans. Because the water-based emulsions according to the invention are stable when repeatedly frozen and thawed, they do not degrade during transport or storage under conditions that expose them to intermittent freezing. In accordance with this discovery, this invention provides safe, stable, micro-emulsion formulations of steroid pheromones that can be administered to mammals, including pigs, as liquids, droplets, pastes or treated inert substrates, but particularly as an aerosol from a hand-held atomizer.

In general terms, the invention is directed to a method of formulating steroid pheromones, including (but not limited to) 5α-androst-16-en-3-one, 3α-hydroxy-5α-androst-16-ene, 5β-androst-16-en-3-one, and Δ4,16-androstadien-3-one, as micro-emulsions prepared using hydrotropes that include (but are not limited to) methanol, ethanol, and all possible isomers of propanol, butanol, pentanol, hexanol, propanediol, butanediol, pentanediol and hexanediol, or any combination of two or more of said hydrotropes, and surfactants that include (but are not limited to) alkylated aryl sulfonic acids, especially dodecyl benzene sulfonic acid, amine-neutralized alkylated arylsulfonates, especially dodecylbenzene sulfonic acid neutralized with monoisopropyl amine, ethoxylated alkylphenols, especially nonylphenols ethoxylated with 9 moles of ethylene oxide, and unsubstituted fatty esters of a polyoxyalkated sorbitan, especially sorbitan mono-oleate ethoxylated with 20 moles of ethylene oxide.

This invention also pertains to compositions in which androstene steroid pheromones, including (but not limited to) 5α-androst-16-en-3-one, 3α-hydroxy-5α-androst-16-ene, 5β-androst-16-en-3-one, and Δ4, 16-androstadien-3-one, are formulated in effective ajmounts as water-based micro-emulsions that include one or more hydrotropes selected from (but not limited to) methanol, ethanol, and all possible isomers of propanol, butanol, pentanol, hexanol, propanediol, butanediol, pentanediol and hexanediol, and one or more surfactants selected from (but not limited to) alkylated aryl sulfonic acids, especially dodecyl benzene sulfonic acid, amine-neutralized alkylated arylsulfonates, especially dodecylbenzene sulfonic acid neutralized with monoisopropyl amine, ethoxylated alkylphenols, especially nonylphenols ethoxylated with 9 moles of ethylene oxide, and unsubstituted fatty esters of a polyoxyalkated sorbitan, especially sorbitan mono-oleate ethoxylated with 20 moles of ethylene oxide. These micro-emulsion compositions can be delivered to mammals, including pigs, as aerosols, liquids, droplets, pastes or as treated inert substrates. When delivered as aerosols, they can be dispensed from a hand-held atomizer. These aerosols can be used to diagnose the onset of sexual maturity in gilts and the timing of oestrus in sows. Other potential uses of these micro-emulsion compositions are to accelerate reproductive maturation in gilts, and to treat dummy sows to train naive boars to mount them for semen collection.

### EXAMPLE 1

The steroid pheromone androstenone was pre-mixed with a mono-alcohol by dissolving 1.52 g of androstenone in 157 g of 2-propanol. The di-alcohol 1,4-butanediol was pre-mixed with a surfactant by dissolving 500 g of dodecylbenzene sulfonic acid as its monoisopropylamine salt in 3,000 g of 1,4-butanediol. The two pre-mixes were then mixed together with constant stirring, while a quantity of water was added in a slow stream in an amount (96,341.48 g) sufficient to attain 100% by weight, to produce a micro-emulsion.

When exposed to room temperature for 150 days, the resulting micro-emulsion was stable, as determined by gas chromatographic analysis. Unexpectedly, this micro-emulsion was also stable after three cycles of freezing for 22-24 h at −15° C., and thawing at room temperature for 2 h, as determined by gas chromatographic analysis.

### EXAMPLE 2

The steroid pheromone androstenone was pre-mixed with a mono-alcohol by dissolving 0.76 g of androstenone in 78.5 g of 2-propanol. The di-alcohol 1,4-butanediol was pre-mixed with a surfactant by dissolving 500 g of dodecylbenzene sulfonic acid in 3,000 g of 1,4-butanediol. The two pre-mixes were then mixed together with constant stirring, while a quantity of water was added in a slow stream in an amount (96,420.74 g) sufficient to attain 100% by weight, to produce a micro-emulsion.

When exposed to room temperature for 150 days, the resulting micro-emulsion was stable, as determined by gas chromatographic analysis. Unexpectedly, this micro-emulsion was also stable after three cycles of freezing for 22-24 h at −15° C., and thawing at room temperature for 2 h, as determined by gas chromatographic analysis.

### EXAMPLE 3

The steroid pheromone androstenone was pre-mixed with a mono-alcohol by dissolving 1.52 g of androstenone in 157 g of 2-propanol. The di-alcohol 1,4-butanediol was pre-mixed with the surfactant by dissolving 500 g of nonylphenol ethoxylated with 9 moles of ethylene oxide in 3,000 g of 1,4-butanediol to produce a micro-emulsion. The two pre-mixes were then mixed together with magnetic stirring, while a quantity of water was added in a slow stream in an amount (96,341.48 g) sufficient to attain 100% by weight.

Twenty mL of the resulting micro-emulsion were placed in a 30 mL high-density polyethylene bottle (Ampak Distribution Inc., Richmond, BC). The bottle was fitted with a mechanical atomizer, which delivered approximately 0.13 mL of an aerosol containing ca. 2 μg of androstenone when the plunger was depressed manually. Two depressions delivered about 4 μg, well within the effective dose of 2-20 μg established by Melrose et al. (1972). When tested against sows by veterinarians, the water-based micro-emulsion composition induced a mating stance at a rate comparable to the available commercial product formulated with an organic solvent and a pressurized propellant.

As will be apparent to those skilled in the art in the light of the foregoing disclosure, many alterations and modifications are possible in the practice of this invention without departing from the spirit or scope thereof. Accordingly, the scope of the invention is to be construed in accordance with the substance defined by the following claims.

### REFERENCES

US Patent Documents

Berliner, D. L. and L. Monti-Bloch. 2001. Steroids as neurochemical stimulators of the VNO to alleviate pain. U.S. Pat. No. 6,331,534.
Cutler, W. B., G. Preti and G. R. Huggins. 1987. Axillary androstenol and dehydroepiandrosterone as fertile period onset indicators. U.S. Pat. No. 4,670,401.
Cutler, W. B., G. Preti and G. R. Huggins. 1989. Birth control method involving monitoring of axillary androstenol and dehydroepiandrosterone. U.S. Pat. No. 4,879,244.
Cutler, W. B., G. Preti and G. R. Huggins. 1990. Axillary androstenol and dehydroepiandrosterone as fertile period onset indicators. U.S. Pat. No. 4,931,403.
Cutler, W. B., G. Preti and C. R. Garcia. 1992. Use of male essence to alter female endocrine response. U.S. Pat. No. 5,155,045.
Jennings-White, C. L., D. L. Berliner, N. W. Adams and L. Monti-Bloch. 2000a. Steroids as neurochemical stimulators of the VNO to alleviate symptoms of PMS and anxiety. U.S. Pat. No. 6,057,439.
Jennings-White, C. L., D. L. Berliner, N. W. Adams and L. Monti-Bloch. 2000b. Steroids as neurochemical stimulators of the VNO to treat paroxistic tachycardia. U.S. Pat. No. 6,117,860.
Melrose, D. R., R. L. S. Patterson and H. C. Reed. 1972. Artificial insemination of pigs. U.S. Pat. No. 3,681,490.
   Other Publications
Antec International. 1996. Eberduft-Spray BOAR MATE (label).
Benton, D. and V. Wastell. 1986. Effects of androstenol on human sexual arousal. Biol. Psych. 22: 141-147.
Brooksbank, B. W. L., R. Brown and J. A. Gustafsson. 1974. The detection of 5α-androst-16-en-3α-ol in human male axillary sweat. Experientia 30: 864-865.
Claus, R. and W. Alsing. 1976. Occurrence of 5α-androst-16-en-3-one, a boar pheromone, in man and its relationship to testosterone. J. Endocrinol. 68: 483-484.
Cowley, J. J. and B. W. L. Brooksbank. 1991. Human exposure to putative pheromones and changes in aspects of social behaviour. J. Steroid Biochem. Molec. Biol. 39: 647-659.
Estienne, M. and A. Harper. 2001. Techniques for training boars for semen collection. Virginia Cooperative Extension, Virginia Polytechnic Institute and State University. http://www.thepigsite.com.
Filsinger, E. E., J. J. Braun, W. C. Monte and D. E. Linder. 1984. Human (Homo sapiens) responses to the pig (Sus scrofo) sex pheromone 5 alpha-androst-16-en-3-one. J. Comp. Psych. 98: 219-222.
Glei, M., W. Schlegel, D. Straube und B. Blankenburg. 1989. Untersuchungen zur Beeinflussung des Pubertatseintrittes von Jungsauen mittels maskuliner Stimuli. Arch. Tierz. 32: 173-179.
Gower, D. B., F. A. Harrison and R. B. Heap. 1970. The identification of C₁₉-16-unsaturated steroids and estimation of 17-oxosteroids in boar spermatic vein plasma and urine. J. Endocrinol. 47: 357-368.
Hoover, J. E. (Ed.). 1975. Remington's pharmaceutical Sciences. 15th ed. Mack Pub. Co., Easton, Pa.
Kirkwood, R. N, P. E. Hughes and W. D. Booth. 1983. The influence of boar-related odours on puberty attainment in gilts. Anim. Prod. 36: 131-136.
Kwan, T. K., D. J. H. Trafford, H. L. J. Makin, A. I. Mallet and D. B. Gower. 1992. GC-MS studies of 16-androstenes and other C₁₉ steroids in human semen. J. Steroid Biochem. Molec. Biol. 43: 549-556.
Labows, J. N., G. Preti, E. Hoelzle, J. Leyden and A. M. Klingman. 1979. Steroid analysis of human apocrine secretion. Steroids 34: 249-258.
Melrose, D. R., H. C. B. Reed and R. L. S. Patterson. 1971. Androgen steroids associated with boar odour as an aid to the detection of oestrus in pig artificial insemination. Br. Vet. J. 127: 497-502.
Nixon, A., A. I. Mallet and D. B. Gower. 1988. Simultaneous quantification of five odorous steroids (16-androstenes) in the axillary hair of man. J. Steroid Biochem. Molec. Biol. 29: 505-510.
Patterson, R. L. S. 1968a. 5α-Androst-16-en-3-one: compound responsible for taint in boar fat. J. Sci. Fd. Agric. 19: 31-38.
Patterson, R. L. S. 1968b. Identification of 3α-hydroxy-5α-androst-16-ene as the musk odour component of boar submaxillary salivary gland and its relationship to the sex odour taint in pork meat. J. Sci. Fd. Agric. 19: 434-438.
Preti, G. and C. J. Wysocki. 1999. Human pheromones: releasers or primers: fact or myth. In: R. E. Johnstone, D. Meuller-Schwarze and P. W. Sorensen (eds.) Advances in chemical signals in vertebrates. Plenum, N.Y. Pp. 315-331.
Preti, G., W. B. Cutler, C. M. Christensen, H. Lawley, G. R. Huggins and C.-R. Garcia. 1987. Human axillary extracts: analysis of compounds from samples which influence menstrual timing. J. Chem. Ecol. 13: 717-731.
Reed, H. C. B., D. R. Melrose and R. L. S. Patterson. 1974. Androgen steroids as an aid to the detection of oestrus in pig artificial insemination. Br. Vet. J. 130: 61-67.
Shinohara, K., M. Morofushi, T. Funabashi, D. Mitsushima and F. Kimura. 2000. Effects of 5α-androst-16-3α-ol on the pulsatile secretion of lutenizing hormone in human females. Chem. Senses 25: 465-467.
Shinohara, K., M. Morofushi, T. Funabashi and F. Kimura. 2001. Axillary pheromones modulate pulsatile LH secretion in humans. Neuroreport 12: 893-895.

What is claimed is:

1. A method of formulating a water-based steroid pheromone containing emulsion comprising:
(a) mixing a steroid pheromone with a suitable hydrotrope to form a first mixture;
(b) mixing a suitable hydrotrope with a suitable surfactant to form a second mixture; and
(c) mixing the first mixture with the second mixture while adding a suitable amount of water to form the water-based emulsion.

2. The method of claim 1, wherein the emulsion is a micro-emulsion.

3. The method of claim 1, wherein the steroid pheromone is an androstene pheromone selected from the group consisting of one or more of: (a) a C₁₉-16 unsaturated androgen steroid, and (b) a C₁₉-4,16 unsaturated androgen steroid.

4. The method of claim 3 wherein the C₁₉-16 unsaturated androgen steroid is selected from the group consisting of 5α-androst-16-en-3-one, 3α-hydroxy-5α-androst-16-ene and 5β-androst-16-en-3-one; and the C₁₉-4,16 unsaturated androgen steroid is Δ4,16-androstadien-3-one.

5. The method of claim 1 wherein the hydrotropes are a combination of mono-alcohol and di-alcohol.

6. The method of claim 1, wherein the hydrotropes are selected from the group consisting of methanol, ethanol, propanol, butanol, pentanol, hexanol, propanediol, butanediol, pentanediol and hexanediol, and isomers thereof, and any combination of two or more of the hydrotropes.

7. The method of claim 1, wherein the surfactant is selected from the group consisting of alkylated aryl sulfonic acids, amine-neutralized alkylated arylsulfonates, ethoxylated alkylphenol and unsubstituted fatty esters of a polyoxyalkated sorbitan.

8. The method of claim 1, wherein the surfactant is selected from the group consisting of dodecyl benzene sulfonic acid, dodecylbenzene sulfonic acid neutralized with monoisopropyl amine, nonylphenols ethoxylated with 9 moles of ethylene oxide, and sorbitan mono-oleate ethoxylated with 20 moles of ethylene oxide.

9. The method of claim 4 wherein the proportions by weight (% w/w) are: mono-alcohol 0.05-0.2, di-alcohol 1-10, steroid 0.0001-0.1, surfactant 0.1-0.5, and water quantity sufficient to bring the composition to 100% w/w.

10. The method of claim 6 wherein the proportions by weight (% w/w) are: mono-alcohol 0.05-0.2, di-alcohol 1-10, steroid 0.0001-0.1, surfactant 0.1-0.5, and water quantity sufficient to bring the composition to 100% w/w.

11. A composition comprising a water-based emulsion formed from a steroid pheromone, one or more hydrotropes, a surfactant and water.

12. The composition of claim 11, wherein the emulsion is a micro-emulsion.

13. The composition of claim 11, wherein the steroid pheromone is an androstene steroid pheromone selected from the group consisting of (a) a C₁₉-16 unsaturated androgen steroid; and (b) a C₁₉-4,16 unsaturated androgen steroid.

14. The composition of claim 13, wherein the C₁₉-16 unsaturated androgen steroid is selected from the group consisting of 5α-androst-16-en-3-one, 3α-hydroxy-5α-androst-16-ene and 5β-androst-16-en-3-one; and the C₁₉-4,16 unsaturated androgen steroid is Δ4,16-androstadien-3-one.

15. The composition of claim 11 wherein the hydrotropes are a combination of mono-alcohol and di-alcohol.

16. The composition of claim 11, wherein the hydrotropes are selected from one or more of the group consisting of methanol, ethanol, propanol, butanol, pentanol, hexanol, propanediol, butanediol, pentanediol and hexanediol and isomers thereof, and combinations of two or more of said hydrotropes.

17. The composition of claim 11, wherein the surfactant is selected from the group consisting of alkylated aryl sulfonic acids, amine-neutralized alkylated arylsulfonate, ethoxylated alkylphenol and unsubstituted fatty esters of a polyoxyalkated sorbitan.

18. The composition of claim 11, wherein the surfactant is selected from the group consisting of dodecyl benzene sulfonic acid, dodecylbenzene sulfonic acid neutralized with monoisopropyl amine, nonylphenols ethoxylated with 9 moles of ethylene oxide, and sorbitan mono-oleate ethoxylated with 20 moles of ethylene oxide.

19. A method of applying a steroid pheromone emulsion composition to a substrate comprising dispensing the emulsion composition claimed in claim 8 as an effective amount of an aerosol, a liquid, droplets or a paste.

20. The method of claim 19, wherein the composition is an aerosol dispensed from an aerosol dispensing device.

21. The method of introducing a steroid pheromone emulsion composition as claimed in claim 11 into the environment of a mammal as an effective amount of an aerosol, liquid, droplets, paste, or treated inert substrate.

22. The method of claim 21, wherein the mammal is a pig.

23. The method of claim 22, wherein the aerosol treatment is used in effective amount as an aid in diagnosing the onset of oestrus in gilts.

24. The method of claim 22, wherein the aerosol treatment is used in effective amount as an aid in diagnosing the timing of oestrus in sows.

25. The method of claim 22, wherein the aerosol, liquid, droplets, paste, or treated inert substrate treatment is used in an effective amount as an aid in accelerating the onset of puberty in gilts.

26. The method of claim 22, wherein the inert substrate is a dummy sow used during collection of semen from a boar.

27. The method of claim 19, wherein the emulsion composition is comprised of one or more steroids selected from the group consisting of C₁₉-16 unsaturated androgen steroids, and C₁₉-4,16 unsaturated androgen steroids; hydrotropes selected from the group consisting of methanol, ethanol, propanol, butanol, pentanol, hexanol, propanediol, butanediol, pentanediol, hexanediol, and any isomers thereof, any combination of two or more of said hydrotropes, surfactants selected from alkylated aryl sulfonic acids, the group consisting of amine-neutralized alkylated arylsulfonates, ethoxylated alkylphenols, and unsubstituted fatty esters of a polyoxyalkated sorbitan.

28. The method of claim 19 wherein the C₁₉-16 unsaturated androgen steroid is selected from the group consisting of 5α-androst-16-en-3-one, 3α-hydroxy-5α-androst-16-ene, and 5β-androst-16-en-3-one; the C₁₉-4,16 unsaturated androgen steroid is Δ4,16-androstadien-3-one, and the surfactant is selected from the group consisting of dodecyl benzene sulfonic acid; dodecyl benzene sulfonic acid neutralized with monoisopropyl amine; nonylphenols ethoxylkated with 9 moles of ethylene oxide; and sorbitan mono-oleate ethoxylated with 20 moles of ethylene oxide.

29. The method of claim 27, wherein the composition is a micro-emulsion.
